(19) United States
(12) Patent Application Publication
(10) Pub. No.: US 2017/0311387 A1
(43) Pub. Date: Oct. 26, 2017

(54) BED SHEET HEATING PAD

(71) Applicant: Wei Hsu, New Taipei City (TW)

(72) Inventor: Wei Hsu, New Taipei City (TW)

(21) Appl. No.: 15/133,235

(22) Filed: Apr. 20, 2016

(51) Int. Cl. H05B 3/34; G08C 17/02; A61B 5/024; A61F 7/00; H05B 1/02; H04L 29/08

(52) U.S. Cl. CPC H05B 3/342; H05B 1/0272; H04L 67/12; A61B 5/024; A61F 7/0097; G08C 17/02

(57) ABSTRACT

A bed sheet heating pad comprises a plurality of control area, each control area comprising a thermal controlling unit, a heating unit, a pressure/temperature sensing unit, a communication unit and a power source, the thermal controlling unit is for receiving control signals from the communication unit to control the heating unit to generate heat, the pressure/temperature sensing unit is for sensing the pressure/temperature of the heating unit and transmitting the sensing signal to the thermal controlling unit to control the temperature of the heating unit, the communication unit is connected to a central control unit and receiving control signal from the central control unit to control the temperature of the control area, and the power source is for supplying the power used in each unit.

### BACKGROUND OF THE INVENTION

### Field of the Invention

The present invention relates to a bed sheet heating pad, and more particularly to an intelligent bed sheet heating pad with multiple heating areas which can be controlled remotely by a computer application.

### The Prior Arts

The mechanism working behind the bed sheet heating pad is transforming the electrical power to thermal energy to deliver the effect of heat preservation. A traditional bed sheet heating pad is relatively simple, basically, it includes a bed sheet cover and a heating unit, and the heating unit is wrapped by the bed sheet cover. The one end of the heating unit is connected to an electronic board, and to another end thereof an electrical power source is connected. The electronic board is used for power on/off the heating pad and for controlling the temperature thereof. Normally, the power source used for the heating pad is not particular but a regular one used in the home. When in use, the electronic board converts the alternating current (AC) into direct current (DC) and the heating unit receives the direct current to generate heat. However, such way the traditional heating pad delivering its heating function is crude and out of date. The heating unit used in the traditional heating pad basically is a piece of plate, and generates heat all over the whole plate, not capable of generating heat in a specified area thereon. Since the limitation of use, the traditional heating pad has some shortcomings, first, the temperature of the heating pad is controlled manually and can't be adjusted automatically based on various situation of the environment. More, when being used for elder people or patient under surveillance, the setting of the heating pad can't be achieved remotely from a remote site.

In view of the above stated, the motivation of the present invention is to provide an intelligent bed sheet heating pad, and some of the areas of which can be specified to generate heat and can be controlled by a computer system remotely from a remote site.

### SUMMARY OF THE INVENTION

The primary object of the present invention is to provide a bed sheet heating pad which comprises a plurality of control area, each control area comprising a thermal controlling unit, a heating unit, a pressure/temperature sensing unit, a communication unit and a power source. The thermal controlling unit is for receiving control signals from the communication unit to control the heating unit to generate heat, the pressure/temperature sensing unit is for sensing the pressure/temperature of the heating unit and transmitting the sensing signal to the thermal controlling unit to control the temperature of the heating unit. The communication unit is connected to a central control unit and receiving control signal from the central control unit to control the temperature of the control area, and the power source is for supplying the power used in each unit. With the bed sheet heating pad of the present invention, a bed sheet heating pad system can be build, the system includes not only every units used in the above bed sheet heating pad but also a central control unit which includes a divisional communication unit connected to the plurality of control area. The central control unit is further connected to a cloud service unit to which a user's terminal device can be connected to transmit an instruction to the central control unit in order to control the plurality of the control area.

### BRIEF DESCRIPTION OF THE DRAWINGS

The related drawings in connection with the detailed descriptions of the present invention to be made later are described briefly as follows, in which:

FIG. 1 is a schematically perspective view of the bed sheet heating pad according to the present invention.

FIG. 2 is a schematically perspective view of the bed sheet heating pad system using the bed sheet heating pad described in FIG.1 according to the present invention.

FIG. 3 is a schematically perspective view illustrating a user is using the bed sheet heating pad of the present invention.

FIG. 4 is a schematically perspective view illustrating a user is using a mobile device via a cloud service unit to control the bed sheet heating bed according to the present invention.

### DETAILED DESCRIPTION OF THE PREFERRED EMBODIMENT

The aforementioned and other objectives and advantages of the present invention will become clearer in light of the following detailed description of an illustrative embodiment of this invention described in connection with the drawings. It is intended that the embodiments and drawings disclosed herein are to be considered illustrative rather than restrictive.

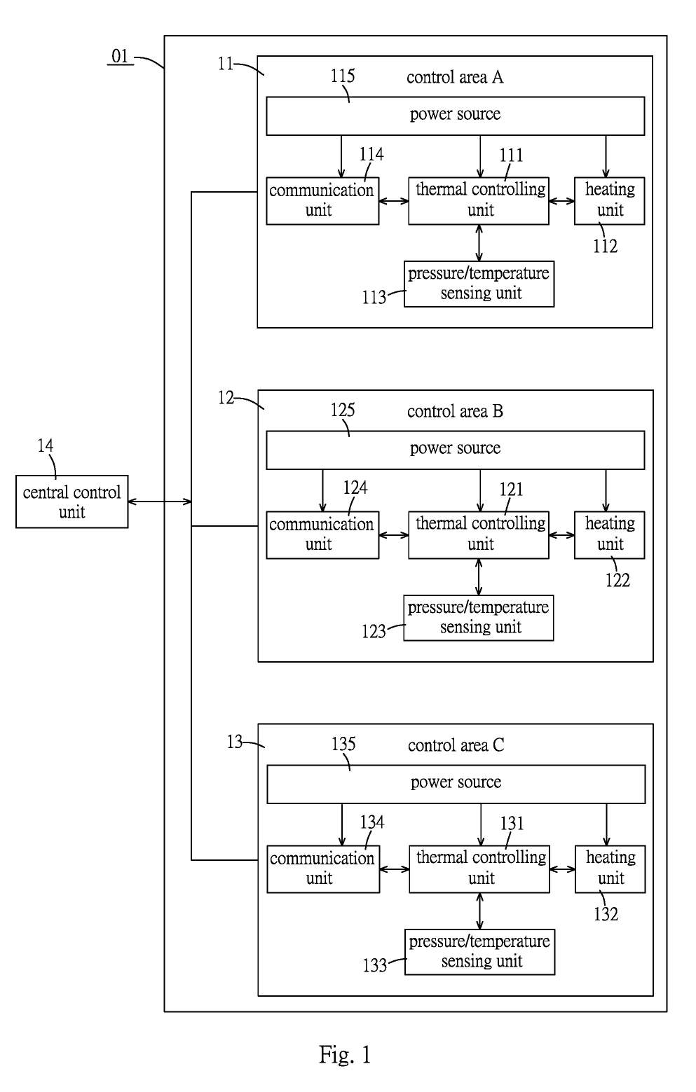

Please Refer to FIGS. 1, FIG. 1 is a schematically perspective view of the bed sheet heating pad according to the present invention. As shown in FIG. 1, the bed sheet heating pad 10 according to the present invention comprises a plurality of control area 11(12, 13), each control area 11(12, 13) comprises a thermal controlling unit 111(121,131), a heating unit 112(122, 132), a pressure/temperature sensing unit 113(123, 133), a communication unit 114(124,134) and a power source 115(125, 135). The thermal controlling unit 111(121,131) is for receiving control signals from the communication unit 114(124,134) to control the heating unit 112(122,132) to generate heat, the pressure/temperature sensing unit 113(123, 133) is for sensing the pressure/temperature of the heating unit 112(122, 132) and transmitting the sensing signal to the thermal controlling unit 111(121,131) to control the temperature of the heating unit. The communication unit 114(124, 134) is connected to a central control unit 14 and receiving control signal from the central control unit 14 to control the temperature of the control area 11(12,13), and the power source 15 is for supplying the power used in each unit.

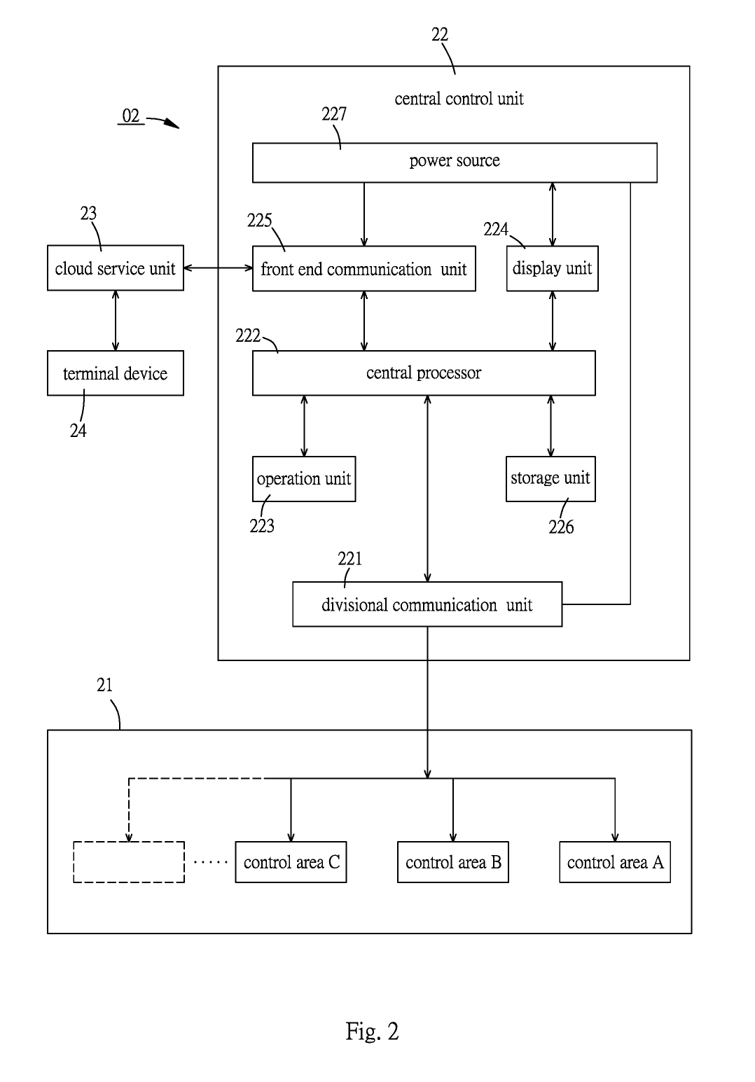

Please Refer to FIGS. 2, FIGS. 2 is a schematically perspective view of the bed sheet heating pad system using the bed sheet heating pad described in FIG.1 according to the present invention. As shown in FIG. 2, with the bed sheet heating pad described in FIG. 1 of the present invention, a bed sheet heating pad system 02 can be build. The system includes not only every units used in the above bed sheet heating pad 21 but also a central control unit 22 which includes a divisional communication unit 221 connected to the plurality of control area A(B, C). The central control unit 22 is further connected to a cloud service unit 23 to which a user's terminal device 24, such as mobile phone, notebook computer, tablet computer and personal computer, can be connected to transmit an instruction to the central control unit 22 to control the plurality of the control area A(B,C).

As shown in FIG. 2, the central control unit 22 of the bed sheet heating pad system 02 not only comprises the divisional communication unit 221 connected to the plurality of control area A(B, C), but also a central processor 222 for processing the signals receiving from every units; an operation unit 223, such as keyboard or remote controller for inputting an instruction to operate the system; a display unit 224 for displaying the system status and providing as a user interface; a front end communication unit 225 for connecting a cloud service unit 23, a user is able to use a terminal device 24, such as mobile phone, notebook computer, tablet computer or a personal computer to connect the cloud service unit 23 to transmit a control instruction to control the plurality of the control area A(B, C); a storage unit 226 for storing a heating plan with which an instruction will be summited at a specific time to control the plurality of the control area A(B, C) for heating; and a power source 227 for supplying electrical power for each units for operation.

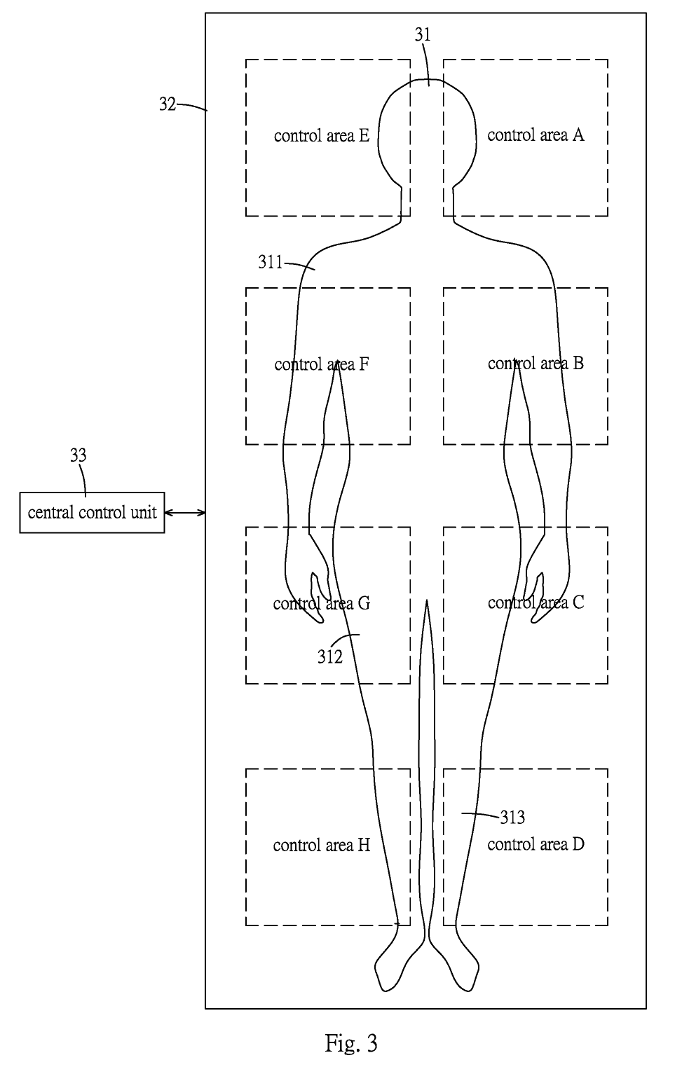

Please refer to FIG. 3, FIG. 3 is a schematically perspective view illustrating a user is using the bed sheet heating pad of the present invention. As shown in FIG. 3, a user 31 is lying on the bed sheet heating pad 32 of the present invention. The user can user the central control unit 33 of the bed sheet heating pad system (element 02 in FIG. 2) to set up the heating temperature and heating period of each control area A(B, C, D, E, F, G, H). For instance, if the user 31 plan to heat his right shoulder 311 at temperature of 15° C. and for 20 minutes, his thigh 312 at temperature of 20° C. and for 30 minutes, his left calf 313 at temperature of 15° C. and for 30 minutes, the user 31 can use the central control unit 33 of the bed sheet heating pad system (element 02 in FIG. 2) to set up the control area F to generate heat at temperature of 15° C. for 20 minutes, the control area G to generate heat at temperature of 20° C. for 30 minutes, the control area D to generate heat at temperature of 15° C. for 30 minutes.

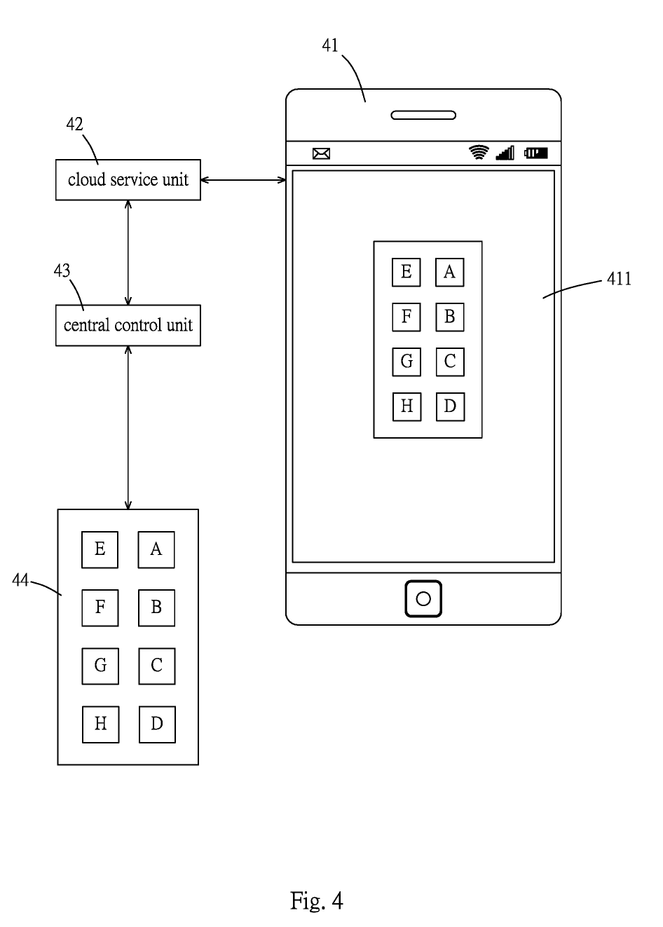

Please refer to FIG. 4, FIG. 4 is a schematically perspective view illustrating a user is using a mobile device via a cloud service unit to control the bed sheet heating pad system according to the present invention. As shown in FIG. 4, if when elder people or patient under surveillance are weak enough to operate the bed sheet heating pad of the present invention alone, his family member is able to use a terminal device, such as mobile phone, notebook computer, tablet computer or personal computer via a cloud service unit to control the heating pad. As shown in FIG. 4, a user is using an application (APP) 411 installed in the mobile phone 41 via the cloud service unit 42 and the central control unit 43 to control the bed sheet heating pad 44 of the present invention. As shown in FIG. 4, the application (APP) 411 installed in the mobile phone 41 can display the plurality of the control area A (B, C, D, E, F, G, H). Double clicking on one of the plurality of the control area A (B, C, D, E, F, G, H) can set up the heating temperature and heating period for that specified area.

As illustrated in the preferred embodiments of the present invention described above, a user can control the heating temperature and heating period of a specified area of the bed sheet heating pad of the present invention. More, the heating temperature and the heating period of a specified area can be changed automatically and flexibly based on the situation of the environment. Such feature is very useful if when elder people or patient under surveillance are weak enough to operate the heating pad alone, his family member is able to use a terminal device, such as mobile phone, notebook computer, tablet computer or personal computer, via a cloud service unit, to control heating temperature and heating period of a specified area of the bed sheet heating pad of the present invention. Additional advantages and modifications will readily occur to those skilled in the art. Therefore, the invention in its broader aspects is not limited to the specific details and representative embodiments shown and described herein. Accordingly, various modifications may be made without departing from the spirit or scope of the general inventive concept as defined by the appended claims and their equivalents.

What is claimed is:

1. a bed sheet heating pad, comprising a plurality of control area, each control area comprising:
a heating unit for generating heat;
a thermal controlling unit for controlling the heating temperature and the heating period of the heating unit;
a pressure/temperature sensing unit for sensing the pressure/temperature of the heating unit and transmitting a sensing signal to the thermal controlling unit to control the temperature of the heating unit;
a communication unit for connecting to a central control unit and receiving a control signal from the central control unit and transmitting the control signal to the thermal controlling unit to control the temperature of the heating unit; and
a power source for supplying electrical power used in each unit.

2. The bed sheet heating pad according to claim 1, wherein said pressure/temperature sensing unit is able to sense a heart beating rate of a user who is using the bed sheet heating pad, and transmit the heart beating rate to the central control unit via the communication unit.

3. The bed sheet heating pad according to claim 1, wherein communication protocol used for said communication unit can be one of the followings: 2.4 GHZ wireless protocol, Zigbee or RS-485.

4. A bed sheet heating pad system, said system comprising:
a bed sheet heating pad;
a central control unit comprising a divisional communication unit and a front end communication unit, said divisional communication unit being connected to the bed sheet heating pad;
a cloud service unit connecting to the central control unit and a terminal device;
wherein, a user is able to use the terminal device, via the a cloud service unit, to connect the central control unit to set up and control the heating temperature and heating period of the bed sheet heating pad.

5. The bed sheet heating pad system according to claim 4, wherein the bed sheet heating pad comprising a plurality of control area, each control area comprising:
a heating unit for generating heat; a thermal controlling unit for controlling the heating temperature and the heating period of the heating unit;
a pressure/temperature sensing unit for sensing the pressure/temperature of the heating unit and transmitting a sensing signal to the thermal controlling unit to control the temperature of the heating unit;
a communication unit for connecting to a central control unit and receiving a control signal from the central control unit and transmitting the control signal to the thermal controlling unit to control the temperature of the heating unit; and
a power source for supplying electrical power used in each unit.

6. The bed sheet heating pad system according to claim 5, wherein said pressure/temperature sensing unit is able to sense a heart beating rate of a user who is using the bed sheet heating pad, and transmit the heart beating rate to the central control unit via the communication unit.

7. The bed sheet heating pad system according to claim 4, wherein communication protocol used for said divisional communication unit can be one of the followings: 2.4 GHZ wireless protocol, Zigbee or RS-485.

8. The bed sheet heating pad system according to claim 4, wherein communication protocol used for said front end communication unit can be one of the followings: USB, LAN, WIFI or BLUETOOTH.

9. The bed sheet heating pad system according to claim 4, wherein said central control unit further comprising:
a central processor for processing the signals receiving from every units;
an operation unit for inputting an instruction to operate the system;
a display unit for displaying the system status and providing as a user interface;
a storage unit for storing a heating plan with which an instruction is summited at a specific time to control the plurality of the control area for heating; and
a power source for supplying electrical power for each units for operation.

10. The bed sheet heating pad system according to claim 4, wherein said terminal device can be one of the followings: mobile phone, notebook computer, tablet 4 computer or personal computer.

11. The bed sheet heating pad system according to claim 9, wherein said operation unit can be a computer keyboard or a remote controller.

12. The bed sheet heating pad system according to claim 9, wherein said display unit can be one of the followings: LED screen, LCD screen or OLED screen.

13. The bed sheet heating pad system according to claim 4, wherein the terminal device has an application (APP) for controlling the bed sheet heating pad for heating.
